(19) United States
(12) Patent Application Publication
(10) Pub. No.: US 2025/0032555 A1
(43) Pub. Date: Jan. 30, 2025

(54) APPLICATION OF PREVOTELLA COPRI IN PREVENTING AND TREATING ATTENTION-DEFICIT-HYPERACTIVITY DISORDER

(71) Applicants: Chongqing Medical University, Chongqing (CN); Children’s Hospital of Chongqing Medical University, Chongqing (CN)

(72) Inventors: Jingfu Qiu, Chongqing (CN); Chengzhi Chen, Chongqing (CN); Yijun Liu, Chongqing (CN); Li Chen, Chongqing (CN); Hongyang Zhang, Chongqing (CN)

(21) Appl. No.: 18/916,729

(22) Filed: Oct. 16, 2024

Related U.S. Application Data
(63) Continuation of application No. PCT/CN2023/119447, filed on Sep. 18, 2023.

(30) Foreign Application Priority Data
Sep. 19, 2022 (CN) 202211135180.5

(51) Int. Cl. A61K 35/741; A61P 25/00

(52) U.S. Cl. CPC A61K 35/741; A61P 25/00

(57) ABSTRACT

An application method of Prevotella copri includes: applying the Prevotella copri in preventing and treating the ADHD. The Prevotella copri is applied in an effective therapeutic amount to achieve treating the ADHD, and a strain number of the Prevotella copri is JCM 13464=DSM 18205. It is indicated through animal experiments that the Prevotella copri can significantly improve hyperactive behavior, spontaneous activity levels and impulsive behavior hyperactivity. A further experimental exploration finds that the Prevotella copri can significantly increase levels of adrenocorticotropic hormone and cortisol in serum, improve neuroendocrine system dysfunction, and significantly increase gene expressions of glucocorticoid receptor (Nr3c1) and mineralocorticoid receptor (Nr3c2), thereby alleviating ADHD social dysfunction, which provides a theoretical basis for application prospects of probiotics in alleviating the ADHD.

### CROSS-REFERENCE TO RELATED APPLICATIONS

This application is a continuation of International Patent Application No. PCT/CN2023/119447, filed on Sep. 18, 2023, which claims the priority of Chinese Patent Application No. CN202211135180.5, filed on Sep. 19, 2022, both of which are herein incorporated by reference in their entirety.

### TECHNICAL FIELD

The disclosure relates to the field of biopharmaceutical technologies, and more particularly to an application of Prevotella copri in preventing and treating attention-deficit hyperactivity disorder (ADHD).

### BACKGROUND

The human gut provides a favorable habitat for microorganisms, the number of microorganisms colonized in the gut is about 10 trillion, which is 10 times the number of human cells, and genes encoded by the microorganisms are 150 times the human genome. As the largest and most complex microecosystem in the human body, gut microbiota and their metabolic byproducts not only can regulate human health, but also play an important role as a bridge between diet and the human body, which are a very important component of the human health system. The gut microbiota can regulate the central nervous system through the microbiota-gut-brain axis, affecting the occurrence and development of neurobehavioral disorders.

ADHD is one of the most common neurodevelopmental disorders in children, with core symptoms of attention deficit and hyperactivity/impulsivity, accompanied by functional impairments in social interaction and academic performance. The prevalence of ADHD in children is approximately 7.2% worldwide, and is 6.26% in China. The ADHD has been considered as a behavioral problem limited to children and adolescents for decades due to the symptoms mostly appearing during preschoolers' school age. However, since the early 1980s, many scholars conduct follow-up studies on the ADHD, and results show that 10%-60% of ADHD patients do not have a resolution of the symptoms, especially social functioning, after adolescence. Instead, the clinical manifestations of the ADHD change, and there are more comorbidities and social problems, which have a significant adverse influence on academic, family and social life of the patients. Therefore, the ADHD increasingly attracts broader attention.

There is evidence to suggest that intestinal immune dysfunction is a part of the pathophysiology of the ADHD. The incidence rate of atopic diseases such as allergic rhinitis, asthma, allergic dermatitis and allergic conjunctivitis increases in hospitalized ADHD patients. In children with the ADHD, the incidence rate of constipation and fecal incontinence is increasing. These findings suggest that intestinal dysbacteriosis may be related to gastrointestinal dysfunction in the ADHD patients and may have an impact on brain function and behavior in these cases.

The gut microbiota of the children with the ADHD shows significant differences in diversity compared to healthy children. A discovery of the microbiota-gut-brain axis confirms that there is a close relationship between the gut microbiota and the nervous system. Gut microbiota can affect the central nervous system by influencing various neurotransmitters, immunity, endocrine and metabolic pathways, thereby causing behavioral and cognitive changes. Restoring the homeostasis of the gut microbiota is a new approach for preventing and alleviating mental and behavioral disorders.

For over 70 years, the treatment of the ADHD mainly uses monoaminergic psychostimulants, primarily methylphenidate (MPH) and amphetamine (AMPH), along with catecholaminergic non-stimulant atomoxetine (ATX). However, in clinical practice, about 20%-30% of patients have a poor or even ineffective treatment with the MPH, unable to achieve symptom control. Additionally, some patients reduce their dosage or even stop taking a medication due to intolerance to side effects of the MPH (e.g., sleep disturbances, decreased appetite, headaches and convulsions, etc.), failing to achieve clinical control. Moreover, some families refuse treatment with central nervous system stimulants, due to limitations of long-term efficacy and a potential risk of abuse of the central nervous system stimulants. Therefore, it is very necessary and urgent to find products with minimal side effects and economical prices, and suitable for preventing the occurrence of the ADHD in sensitive populations.

Prevotella copri belongs to the Bacteroidetes phylum and is a Gram-negative anaerobic bacterium. The Prevotella copri is generally considered a bacterium associated with a healthy plant-based diet, playing a “probiotic” role in the human body. A reduction of the Prevotella copri is associated with certain diseases. Patients diagnosed with dementia with Lewy bodies are found to have lower abundance of the Prevotella copri. A decrease in levels of the Prevotella copri in infants are associated with behavioral problems at two years old. Some dietary research reports indicate that the Prevotella copri is positively correlated with diets rich in polysaccharides and fibers. Furthermore, a negative correlation has been observed between the abundance of the Prevotella copri in patients with first-episode psychosis (FEP). However, there is no research reports on whether the Prevotella copri can improve the symptoms of the ADHD. Probiotic products on the market are mostly aimed at supplementing probiotics without in-depth study of the functions of probiotic strains, greatly underestimating the role of probiotics, and there are no probiotic products related to the ADHD.

### SUMMARY

The disclosure aims at solving at least one of technical problems in the related art to a certain extent. A purpose of the disclosure is mainly to provide an application method of Prevotella copri.

The purpose of the disclosure is achieved through following technical solutions.

The application method of the Prevotella copri includes: applying the Prevotella copri in preventing and treating attention-deficit hyperactivity disorder (ADHD). The Prevotella copri is applied in an effective therapeutic amount to achieve treating the ADHD, and a strain number of the Prevotella copri is Japan Collection of Microorganisms (JCM) 13464=Deutsche Sammlung von Mikroorganismen und Zellkulturen (DSM) 18205.

In an embodiment, the ADHD includes ADHD caused by abnormal brain function development.

In an embodiment, the effective therapeutic amount of the Prevotella copri is 200 microliters (μL), and a concentration of the Prevotella copri is 2.8×10⁸ colony-forming units per milliliter (CFU/mL).

In an embodiment, the Prevotella copri is formulated into a pharmaceutical preparation, the pharmaceutical preparation includes any one selected from the group consisting of a liquid preparation, a solid preparation, a semi-solid preparation, and a gaseous preparation.

In an embodiment, a dosage form of the pharmaceutical preparation includes any one of pharmacologically acceptable dosage forms.

In an embodiment, the pharmaceutical preparation further includes any one or more of pharmaceutically acceptable excipients.

In an embodiment, an administration method for the Prevotella copri includes gavage administration.

Compared to the related art, the disclosure has below beneficial effects.

The disclosure provides a new use for the Prevotella copri, which is applied in preventing and treating of symptoms related to the ADHD. The Prevotella copri is a probiotic strain that alleviates abnormal psychological behavior states associated with the ADHD by balancing gut microbiota and regulating the endocrine dysfunction of the brain-gut axis. Through animal experiments, it is verified that the Prevotella copri significantly improves hyperactive behavior, spontaneous activity levels and impulsive behavior. Further experimental explorations find that the Prevotella copri can significantly increase levels of adrenocorticotropic hormone and cortisol in serum, improve neuroendocrine system dysfunction, and significantly increase the gene expression of a glucocorticoid receptor (nuclear receptor subfamily 3 group C member 1, Nr3c1) and a mineralocorticoid receptor (nuclear receptor subfamily 3 group C member 2, Nr3c2), thereby alleviating social dysfunction in the ADHD and providing a theoretical basis for application prospects of probiotics in alleviating the ADHD.

### BRIEF DESCRIPTION OF DRAWINGS

In order to provide a clearer explanation of embodiments of the disclosure, the accompanying drawings required in descriptions of the embodiments or the related art is briefly introduced below.

FIG. 1 illustrates a growth curve of Prevotella copri in the disclosure.

FIG. 2 illustrates a bacterial solution concentration of the Prevotella copri in the disclosure.

FIG. 3A to FIG. 3E illustrate effects of the Prevotella copri on hyperactivity behavior and spontaneous activity levels in rats with ADHD (spontaneously hypertensive rat, SHR) in the disclosure.

FIG. 4A to FIG. 4E illustrate effects of the Prevotella copri on impulsive behavior in rats with ADHD (SHR) in the disclosure.

FIG. 5A and FIG. 5B illustrate effects of the Prevotella copri on hormones related to the gut-brain axis neuroendocrine system in brain tissues of rats with ADHD (SHR) in the disclosure.

FIG. 6A and FIG. 6B illustrate effects of the Prevotella copri on neuroendocrine system-related gene expression in brain tissue of rats with ADHD (SHR) in the disclosure.

### DETAILED DESCRIPTION OF EMBODIMENTS

In order to make the technical means, creative features, objectives and effects of the disclosure easy to understand, the following embodiments and the accompanying drawings are combined to further elaborate the disclosure. However, the following embodiments are only some preferred embodiments of the disclosure and not all of them.

Experimental methods used in the following embodiments are conventional methods unless otherwise specified. The materials and reagents, etc. used in the following embodiments can be obtained from commercial sources unless otherwise specified.

Prevotella copri of the disclosure is deposited at Shanghai Bioresource Collection Center (SHBCC), a strain number of the Prevotella copri is JCM 13464=DSM 18205.

### Embodiment 1: Cultivation of the Prevotella copri

The Prevotella copri of the disclosure is obtained by the following cultivation method, including following steps.

(1) Activation of the Prevotella copri

The activation of the Prevotella copri is carried out in an anaerobic operating platform. A test tube containing 15 milliliters (mL) of thioglycolate fluid medium [15.0 grams (g) of casein peptone (trypsin hydrolysis), 5.0 g of yeast extract powder, 5.0 g of glucose, 0.5 g of sodium thioglycolate (C₂H₅NaO₂S), 0.5 g of L-cysteine, 2.5 g of sodium chloride (NaCl), 0.001 g of resazurin (C₁₂H₇NO₄), 0.75 g of agar, a potential of hydrogen (pH) of 7.1±0.2] placed in an anaerobic environment for deoxygenation for 24 hours (h) in advance is prepared. After disinfecting the surface of an ampoule containing a lyophilized powder of the Prevotella copri with alcohol, the top of the ampoule is burned with an alcohol lamp and then is quickly moved to an anaerobic incubator to cool to break it. Then, 0.5 mL of the thioglycolate fluid medium is added to the ampoule to fully dissolve the lyophilized powder to obtain a Prevotella copri suspension, the Prevotella copri suspension is added to the test tube containing the thioglycolate fluid medium and then sealed and placed in a sealed anaerobic culture bag of 15×30 centimeters (cm), the sealed anaerobic culture bag is then immediately put into an anaerobic gas generation bag, and then the anaerobic gas generation bag is placed statically in an anaerobic incubator at 37° C. with a humidity of 65% for incubation until the bacteria solution in the test tube becomes turbid or there is a large amount of bacterial growth at the bottom of the test tube, so as to obtain an activated Prevotella copri solution.

(2) Determination of a Growth Curve of the Prevotella copri

A turbidimetric method is used to determine the number of the Prevotella copri and understand growth condition and metabolic status of the Prevotella copri. A relationship between the Prevotella copri suspension and an optical density (OD) value is determined by using a spectrophotometer to clarify a growth pattern of the Prevotella copri, and the OD value is correlated with a cultivation time to establish the growth curve of the Prevotella copri for uniformity of Prevotella copri solution in subsequent in vivo and in vitro experiments.

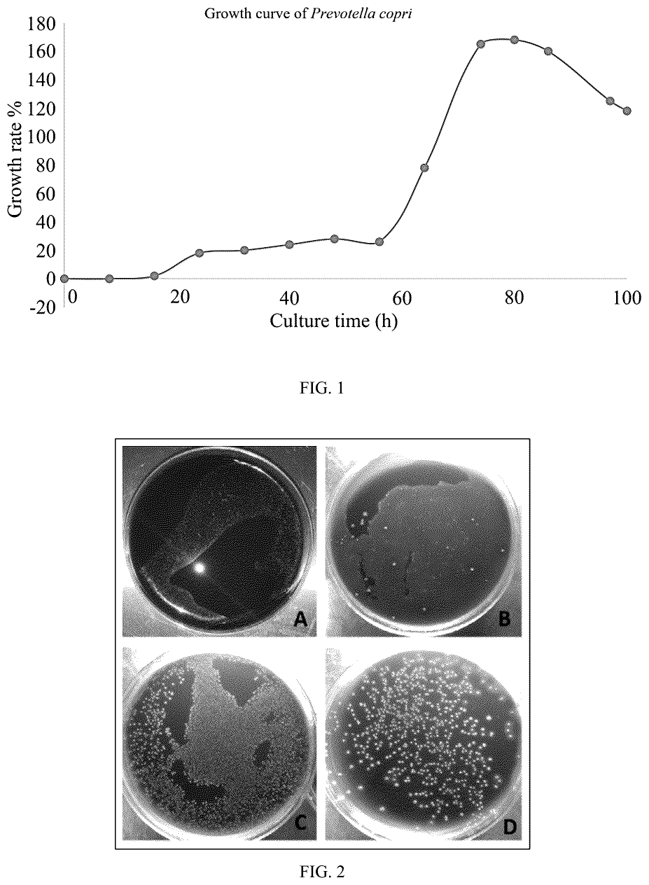

The growth curve of the Prevotella copri is shown in FIG. 1, which illustrates the growth curve of the Prevotella copri. It can be seen in the FIG. 1 that a viable count of the Prevotella copri increases rapidly within 60 h to 72 h. At this period, the Prevotella copri adapts to the culture medium environment with vigorous bacterial community vitality, and begins to grow and reproduce at a high speed, in a logarithmic growth phase.

(3) Calculation of a Concentration of Prevotella copri Solution

0.1 mL of the mixed solution (i.e., the activated Prevotella copri solution obtained in the step (1)) is pipetted into a test tube containing 10 mL of the thioglycolate fluid medium to obtain a Prevotella copri solution at a dilution rate of 10⁻¹. The above process is repeated for 10-fold serial dilution down to a dilution rate of 10⁻⁸. Three dilution rates of 10⁻⁶, 10⁻⁷ and 10⁻⁸ the Prevotella copri solution are selected, and then 0.1 mL of the Prevotella copri solution at each dilution rate is pipetted onto a plate with a Columbia blood agar medium [5.0 g of casein tryptone, 5.0 g of soybean peptone, 10.0 g of the yeast extract powder, 10.0 g of the glucose, 0.5 g of the L-cysteine, 0.001 g of the resazurin, 0.04 g of potassium dihydrogen phosphate (KH₂PO₄), 0.04 g of dipotassium hydrogen phosphate (K₂HPO₄), 0.4 g of sodium bicarbonate (NaHCO₃), 0.08 g of the sodium chloride, 0.008 g of calcium chloride (CaCl₂)), 0.0192 g of magnesium sulfate (MgSO₄), 5% of sterile defibrinated sheep blood, 0.001 g of polymyxin B, and a pH of 7.0], followed by spreading the plate to obtain a coated culture, the coated culture is placed in an incubator at 37° C. overnight for incubation through an anaerobic culture method (5% CO2). After overnight incubation (12-24 h) until colonies grow to be clear and non-coalescent, the number of colonies is counted (n). Based on counting results, a standard curve is drawn and the Prevotella copri solution concentration is calculated.

A calculation formula of the Prevotella copri solution concentration is:

$$N ( CFU / mL ) = n \times \frac{{d}_{2}}{v} ,$$

where n represents an average colony number, d₂ represents the dilution rate, and v represents a volume of Prevotella copri solution coated on the plate.

The Prevotella copri solution concentration of the Prevotella copri is shown in FIG. 2, where A represents colony growth of the mixed solution on the plate with the Columbia blood agar medium after cultivation for 72 h. B represents colony growth of the Prevotella copri solution at the 10⁻⁶ dilution rate on the plate with the Columbia blood agar medium after cultivation for 72 h. C represents colony growth of the Prevotella copri solution at the 10⁻⁷ dilution rate on the plate with the Columbia blood agar medium after cultivation for 72 h. D represents colony growth of the Prevotella copri solution at the 10⁻⁸ dilution rate on the plate with the Columbia blood agar medium after cultivation for 72 h.

(4) Passage Culture and Preservation of the Prevotella copri

10 test tubes containing the thioglycolate fluid medium is prepared, 500 μL of activated Prevotella copri solution is inoculated into the test tubes, and then sealed and placed in a 15×30 cm sealed anaerobic culture bag, the sealed anaerobic culture bag is then placed in an anaerobic gas generation bag. After static cultivation at 37° C. in an anaerobic incubator for 1 day, there is clearly visible turbidity in the thioglycolate fluid medium. Strain cryopreservation tubes and 60% (volume fraction) sterile glycerol are sterilized at high-pressure and placed in the anaerobic incubator, then the Prevotella copri solution and the sterile glycerol are transferred into the strain cryopreservation tubes at a 1:1 ratio. About 1 mL of Prevotella copri suspension is added to each strain cryopreservation tube, making a final glycerol concentration reach 30%, and then stored at −80° C.

(5) Preparation of Prevotella copri Solution

The Prevotella copri reaches the logarithmic growth phase after 72-80 h of cultivation. A pipette is used to absorb 1 mL of the Prevotella copri culture from the logarithmic growth phase into the spectrophotometer cuvette, and the OD₆₀₀ value is measured with an ultraviolet spectrophotometer to determine the Prevotella copri solution concentration. When the OD₆₀₀≈1, the Prevotella copri solution concentration is about 10⁸-10⁹ CFU/mL. After centrifuging the Prevotella copri stock solution at 6000×g for 5 min, the supernatant is removed and the precipitate is resuspended in sterile phosphate-buffered saline (PBS) to prepare a Prevotella copri suspension at about 10⁸-10⁹ CFU/mL. The Prevotella copri suspension is divided into sterilized centrifuge tubes and then sealed to obtain samples. The samples are administered to experimental animals by gavage according to sterile transfer requirements.

### Embodiment 2: Effects Validation and Exploration Experiments of the Prevotella copri

In the following experiments, selected experimental animals are: 16 three-week-old male spontaneously hypertensive rats (SHR), with room temperature maintained at 23±1° C., humidity maintained at 50-60%, a light-dark cycle of 12 h, and free access to clean food and water for acclimation.

Experiment methods: the SHR are divided into two groups: Vehicle (SHR) group and the Prevotella copri gavage group (P. copri), with 8 rats in each group. The Vehicle group is administered by gavage daily with 200 μL of saline solution, the P. copri group is administered by gavage with fermented liquid (containing 10⁸ CFU/mL of live bacteria), the P. copri group is administered daily by gavage with 200 μL of Prevotella copri solution (2×10⁸-2×10⁹ CFU/mL) with an administration time from 8:00 to 9:00 AM, continuously for 14 days. On the 15^{th} day, after a fasting period for 12 h, behavioral experiments are conducted on the rats in each group. On the 16^{th} day, after a fasting period for 12 h, blood is taken from orbital cavity of each rat, and then centrifuged at 3000 revolutions per minute (r/min) to obtain and reserve serum for future use. Brain tissues (cortex, hippocampus) are collected and used for detections such as enzyme-linked immunosorbent assay (ELISA) kit and quantitative polymerase chain reaction (qPCR) method.

Experiment data is represented as mean values±standard error of the mean (SEM). Differences between the two groups are assessed by using independent sample T-tests. All charts are created by GraphPad Prism 8.0 (a software for data analysis and graphics drawing).

I. Effects of the Prevotella copri on Hyperactivity Behavior and Spontaneous Activity Levels

To test the effects of the Prevotella copri on the hyperactive behavior, the spontaneous activity levels and impulsive behavior, the following tests are conducted.

### 1. Open Field Test

The open field test is used to evaluate autonomous activity, active exploration and anxiety state of the rats in a new environment. An open field device is a black box with a length of 50 cm, a width of 50 cm, and a height of 40 cm with an open top. There is a camera connected to a computer above the center of the open field device to record activity information of the rats in the open field device. Before the experiment, open field experiment system parameters are set in the computer, the rats are allowed to adapt to a surrounding environment for 5 min, and then put into the open field device, and activities of the rats in the open field device for 5 min are recorded. After the experiment on each rat, the rat feces and urine in the open field experiment device are cleaned, then the open field device is thoroughly cleaned with 75% acetic acid solution and dried with a paper towel or dry cloth. The activity information recorded includes a total travelled distance, a central travelled distance, a central activity duration and an activity trajectory map.

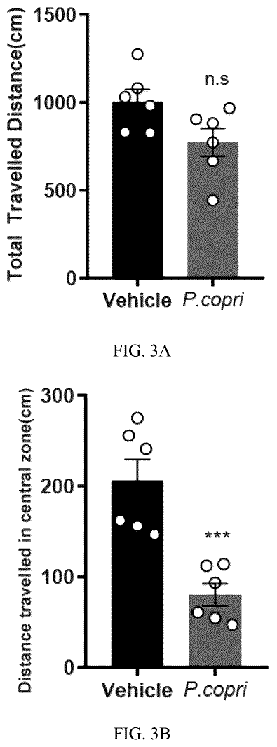

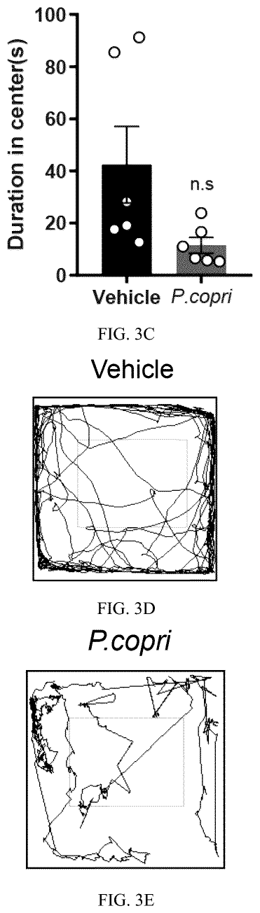

Results are shown as FIG. 3A to FIG. 3E, FIG. 3A to FIG. 3E illustrate the effects of the Prevotella copri on the hyperactivity behavior and spontaneous activity levels in the rats (SHR) with ADHD. FIG. 3A shows that after two weeks of continuous gavage with the Prevotella copri, the total travelled distance of the rats in the P. copri group in the open field test significantly decreases. FIG. 3B shows that after two weeks of continuous gavage with the Prevotella copri, the distance travelled in central zone of the rats in the P. copri group in the open field test significantly decreases. FIG. 3C shows that after two weeks of continuous gavage with the Prevotella copri, the central activity time (i.e., duration in center) of the rats in the P. copri group in the open field test significantly decreases. The data is expressed as mean values±SEM, n=6, *P<0.05; **P<0.01; ***P<0.001. FIG. 3D shows that the activity trajectory map of the rats in the Vehicle group in the open field test. FIG. 3E shows the activity trajectory map of the rats in the P. copri group in the open field test.

### 2. Elevated Plus Maze

The elevated plus maze is a vertical cross formed by two closed walls (50 cm×10 cm), two open walls (50 cm×10 cm), and a central area (10 cm×10 cm), with a distance of 50 cm to the ground. 2 h before the experiment, the rats are placed in a soundproof room to adapt to the environment. The room temperature and ventilation are strictly controlled. The experimenter places a rat at the central area from a same position each time, with the rat's head facing one of the open walls. The experiment begins after the experimenter leaves the experimental device area. The experiment lasts for 5 min, and main indicators for evaluating the impulsive behavior of the rat are a percentage of time that the rat spent in open arms relative to the total time and a percentage of times that the rat enters into the open arms relative to a total number of times.

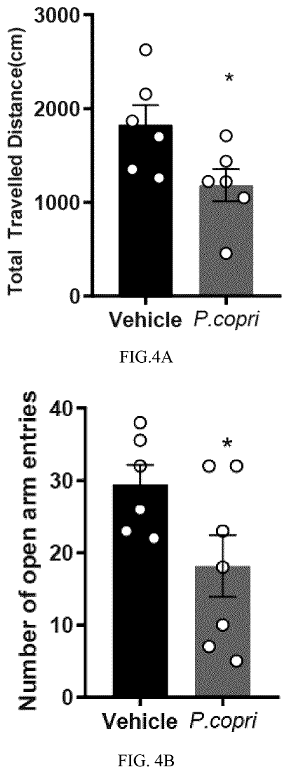

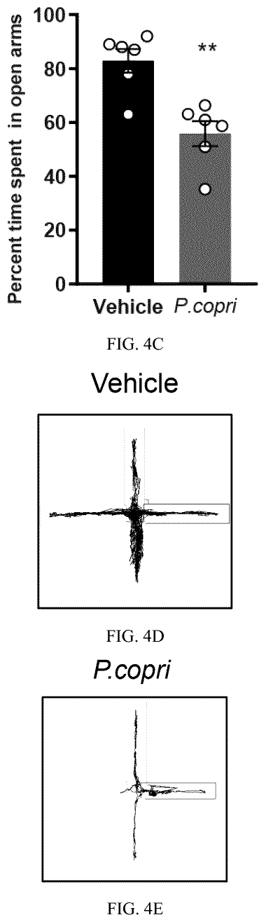

Results are shown as FIG. 4A to FIG. 4E, FIG. 4A to FIG. 4E illustrate the effects of the Prevotella copri on the impulsive behavior in the rats with the ADHD (SHR). FIG. 4A shows that after two weeks of continuous gavage with the Prevotella copri, the total travelled distance of the rats in the P. copri group in the elevated plus maze experiment significantly decreases. FIG. 4B shows that after two weeks of continuous gavage with the Prevotella copri, the number of times that the rats in the P. copri group entered the open arms significantly decreases. FIG. 4C shows that after two weeks of continuous gavage with the Prevotella copri, the percentages of time that the rats in the P. copri group spent in the open arms in the elevated plus maze significantly decrease. The data is expressed as mean values±SEM, n=6, *P<0.05; **P<0.01; ***P<0.001. FIG. 4D shows that the activity trajectory map of the rats in the Vehicle group in the elevated plus maze. FIG. 4E shows the activity trajectory map of the rats in the P. copri group in the elevated plus maze.

II. The Effects of the Prevotella copri on Hormones Related to the Gut-Brain Axis Neuroendocrine System in the Brain Tissues

In response to the serum collected from the experiment methods, ELISA is used to measure levels of the hormones related to the gut-brain axis neuroendocrine system: adrenocorticotropic hormone (ACTH), cortisol (CORT).

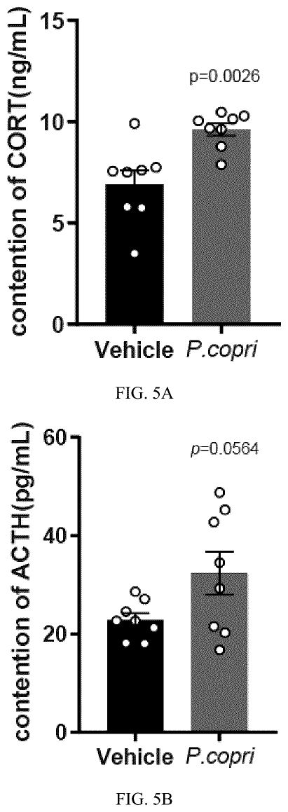

Results are shown in FIG. 5A and FIG. 5B, FIG. 5A and FIG. 5B illustrate the effects of the Prevotella copri on the hormones related to the gut-brain axis neuroendocrine system in the brain tissues of the rats (SHR) with the ADHD. FIG. 5A shows that compared to the rats in the Vehicle group, ACTH levels of the rats treated with the Prevotella copri for two weeks significantly increase in the serum. FIG. 5B shows that the rats treated with the Prevotella copri for two weeks have significantly higher CORT levels in the serum than the rats in the Vehicle group. The data is expressed as mean±SEM, n=6, *P<0.05; **P<0.01; ***P<0.001. The results of the ELISA indicate that the Prevotella copri can improve the endocrine system dysfunction in the ADHD, and regulate the function of the hypothalamic-pituitary-adrenal (HPA) axis.

III. The Effects of the Prevotella copri on Neuroendocrine System-Related Gene Expression in the Gut-Brain Axis of the Brain Tissues

The qPCR method is used to determine expression levels of genes related to the gut-brain axis neuroendocrine system in the brain tissues collected from the experiment methods: glucocorticoid receptor (Nr3c1), mineralocorticoid receptor (Nr3c2).

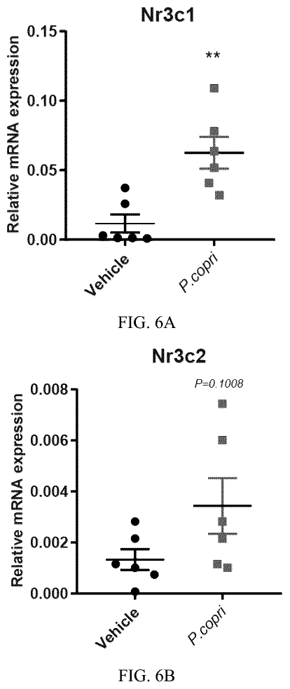

Results are shown in FIG. 6A and FIG. 6B, FIG. 6A shows that the effect of the Prevotella copri on the mRNA expression levels of Nr3c1 in the hippocampus of the rats with the ADHD (SHR). FIG. 6B shows the effect of the Prevotella copri on the mRNA expression levels of Nr3c2 in the hippocampus of the rats with the ADHD (SHR). The Data is represented as mean±SEM, n=6, *P<0.05; **P<0.01; ***P<0.001. After two weeks of gavage with the Prevotella copri solution, the mRNA expression levels of Nr3c1 and Nr3c2 in the brain tissues of the P. copri group are significantly upregulated compared to the Vehicle group. The results indicate that the Prevotella copri may regulate the function of the neuroendocrine system of the brain-gut axis through a certain mechanism, thereby improving ADHD related symptoms.

After administering the Prevotella copri solution to the SHR via gavage for two weeks, their hyperactive behavior, spontaneous activity level, and impulsive behavior are significantly improved. Additionally, hormones and gene expression related to the hypothalamic-pituitary-adrenal (HPA) axis are significantly upregulated. Based on the experimental results, it is speculated that the Prevotella copri may alleviate ADHD symptoms by regulating HPA axis function.

In summary, the disclosure indicates that the Prevotella copri has the potential to alleviate symptoms of the hyperactivity and the impulsive behavior related to the ADHD. The effect may be exerted by regulating the levels of the hormones and gene expression related to neuroendocrine function in the gut-brain axis. The disclosure provides a strong theoretical basis for the therapeutic potential of probiotics in treating the ADHD, and has high social significance and potential market value.

The above embodiments are only used to illustrate the technical solutions of the disclosure, and are not intended to limit them. Although the disclosure has been described in detail with reference to the embodiments, those skilled in the art should understand that they can still make modifications to the technical solutions recorded in the embodiments, or perform equivalent substitutions for some or all of the technical features. The modifications or substitutions do not depart from the essence of the corresponding technical solutions within the scope of the embodiments of the disclosure, and should be covered within the scope of the claims and the description of the disclosure.

What is claimed is:

1. An application method of Prevotella copri, comprising: applying the Prevotella copri in preventing and treating attention-deficit hyperactivity disorder (ADHD);
wherein the Prevotella copri is applied in an effective therapeutic amount to achieve treating the ADHD, and a strain number of the Prevotella copri is Japan Collection of Microorganisms (JCM) 13464=Deutsche Sammlung von Mikroorganismen und Zellkulturen (DSM) 18205.

2. The application method as claimed in claim 1, wherein the ADHD comprises ADHD caused by abnormal brain function development.

3. The application method as claimed in claim 1, wherein the effective therapeutic amount of the Prevotella copri is 200 microliters (μL), and a concentration of the Prevotella copri is 2.8×10⁸ colony-forming units per milliliter (CFU/mL).

4. The application method as claimed in claim 1, wherein the Prevotella copri is formulated into a pharmaceutical preparation, the pharmaceutical preparation comprises any one selected from the group consisting of a liquid preparation, a solid preparation, a semi-solid preparation, and a gaseous preparation.

5. The application method as claimed in claim 4, wherein a dosage form of the pharmaceutical preparation comprises any one of pharmacologically acceptable dosage forms.

6. The application method as claimed in claim 4, wherein the pharmaceutical preparation further comprises any one or more of pharmaceutically acceptable excipients.

7. The application method as claimed in claim 1, wherein an administration method for the Prevotella copri comprises gavage administration.
